(19) United States
(12) Patent Application Publication
(10) Pub. No.: US 2015/0335470 A1
(43) Pub. Date: Nov. 26, 2015

(54) PATIENT WARMING GOWN WITH PERIPHERAL WARMING

(71) Applicant: 3M INNOVATIVE PROPERTIES COMPANY, Saint Paul, MN (US)

(72) Inventors: Carol J. Panser, Minneapolis, MN (US); Albert P. Van Duren, Stillwater, MN (US)

(21) Appl. No.: 14/650,619

(22) PCT Filed: Oct. 30, 2013
(86) PCT No.: PCT/US2013/067475

§ 371 Date: Jun. 9, 2015

Related U.S. Application Data
(60) Provisional application No. 61/736,863, filed on Dec. 13, 2012.

(51) Int. Cl. A61F 7/02; A41D 13/005; A41D 13/12

(52) U.S. Cl. CPC A61F 7/02; A41D 13/1236; A41D 13/0051; A61F 2007/0029

(57) ABSTRACT

A warming device includes a clinical garment with a flexible inflatable convective apparatus integrated with or supported on a surface of the garment for warming one or more limbs when the apparatus is inflated with warmed air. The convective apparatus includes a central member, one or more peripheral diffusers, and one or more flexible ducts. In some cases, each duct connects a diffuser with the central member. One or more inlet ports are provided in the convective apparatus to receive the end of an air hose. The central member is inflatable in response to a flow of air through an inlet port. A peripheral diffuser is positioned on the clinical garment so as to be deployable to or near to an extremity such as a limb. When a peripheral diffuser is deployed while the central member is inflated with warmed air, the warmed air flows from the central member, through a duct to the peripheral diffuser and is emitted through an air-permeable wall of the diffuser to warm the extremity.

### BACKGROUND

The field includes a warming device constituted of a clinical garment with a flexible inflatable convective apparatus supported on an inside surface of the garment for warming peripheral parts of the patient when the apparatus is inflated with warmed air.

Intraoperative hypothermia due to anesthetic redistribution is difficult to treat mainly because it is the result of large internal heat flows from the core to the peripheral thermal compartments and not by the relatively low heat transfer rate from the cutaneous surface. For this reason, patient warming before surgery (“prewarming”) with inflatable convective thermal devices is used to prevent hypothermia due to redistribution during surgery. Examples of such devices include inflatable thermal blankets such as are sold by Arizant Healthcare Inc. under the BAIR HUGGER® brand. It is often desirable that the thermal device used for prewarming also be available to warm the patient during and after surgery. However, during intraoperative use, thermal blankets must be removed or rearranged to allow access to the operative field. Typically, if the patient is hypothermic following surgery, another thermal blanket must be used to re-establish normothermia.

Recently-developed perioperative convective warming technology has increased the versatility of convective clinical warming by combining inflatable convective apparatuses with clinical garments. Examples include patient warming gowns such as are sold by Arizant Healthcare Inc. under the BAIR PAWS® brand. In this regard, a “patient warming gown” is a warming device constituted of a clinical gown equipped with at least one inflatable convective warming apparatus attached to the gown. A patient wearing the gown is warmed by circulation of thermally-conditioned air emitted into the gown from a convective apparatus. Patient warming gowns are exemplified by the warming devices described and claimed in U.S. Pat. Nos. 7,226,454; 7,276,076; 7,364,584; 7,819,911; 7,846,192; 7,862,599; 8,070787; and, 8,192,475.

Patient warming gowns can simplify the perioperative warming process and save money by eliminating the costs and procedures of buying, storing and using multiple items like cotton gowns, cotton blankets, and inflatable thermal blankets. A single patient warming gown can warm many types of medical procedures, including those involving upper body, torso, or lower body. Nevertheless, there is still a need for greater versatility in warming more specialized procedures, such as those involving thoracic, abdominal, or pelvic access.

### SUMMARY

A patient warming gown with peripheral warming is constituted of a clinical garment with a convective apparatus supported on an inside surface of the garment for warming one or more limbs when the apparatus is provided with warmed air. The convective apparatus includes a central member, one or more peripheral diffusers, and one or more non-kinking flexible ducts. Each duct connects a peripheral diffuser with the central member. One or more inlet ports are provided in the convective apparatus to receive the end of an air hose.

The central member of the convective apparatus is attached to the inside surface of the clinical garment. An inlet port opens through the garment into space within the central member. The diffusers may be folded, rolled, or contracted and retained on the garment and then unfolded or unrolled or expanded and deployed for operation when needed.

The convective apparatus is constituted of flexible fabric parts which are assembled to form inflatable structures in the central member, the diffusers, and the ducts. In response to a stream of pressurized air flowing into the convective apparatus through an inlet port, the central member inflates. In some aspects, the central member has a permeable wall and emits air through the air-permeable wall when inflated. When a diffuser is unfolded, unrolled, or expanded, air flowing from the central member, through a duct to the diffuser, inflates both of those parts and is emitted through an air-permeable wall in the diffuser.

A patient dons the patient warming gown. An air hose coupled to an inlet port conducts a stream of warmed pressurized air into the inflatable structure in the central member, causing the central member to inflate. If the ducts and diffusers are folded, rolled, or contracted and retained, the patient is warmed principally by the central member. Alternatively, one or more of the peripheral diffusers can be released, unfolded and deployed to also warm the patient peripherally.

### BRIEF DESCRIPTION OF THE DRAWINGS

FIG. 1 is an illustration of a person wearing a patient warming gown.

FIGS. 2A and 2B are perspective views of a clinical garment for a patient warming gown, with the clinical garment opened to show an inside surface. FIG. 2A shows the clinical garment without a convective apparatus; FIG. 2B shows the clinical garment with a convective apparatus.

FIGS. 3A and 3B are plan views of a convective apparatus for a patient warming gown. FIG. 3A shows the elements of the convective apparatus with peripheral diffusers unfolded. FIG. 3B shows details of folding elements of the peripheral diffusers.

FIGS. 4A, 4B, and 4C are plan, side sectional, and transverse sectional views, respectively, of a pleated duct.

FIG. 5 illustrates deployment of a patient warming gown with provision for peripheral warming of a patient.

FIG. 6 illustrates deployment of the patient warming gown of FIG. 5 with the clinical garment operated to expose abdominal and pelvic areas of a patient.

FIG. 7 illustrates deployment of the patient warming gown of FIG. 5 with the clinical garment operated to expose pelvic and upper leg areas of a patient in a Fowler position.

### DETAILED DESCRIPTION OF THE PREFERRED EMBODIMENTS

A device for warming the body of a person to provide thermal therapy and/or thermal comfort includes a clinical garment and at least one convective apparatus supported on an inside surface of the garment. In this regard, a “clinical garment” is a garment that is typically used to temporarily clothe a patient in a clinical setting. Examples of a clinical garment include patient gowns, hospital gowns, examination gowns, and other equivalents. The clinical setting may be a medical or dental office or clinic, a hospital, or any facility or institution that provides medical or dental treatment to patients. A convective apparatus receives and distributes at least one stream of warmed pressurized air in a structure for being disposed on, adjacent, or next to the core and/or the limbs of a body. When pressurized with warmed air, a convective apparatus emits warmed air through one or more permeable walls.

The preferred clinical garment is a hospital gown. With at least one convective warming apparatus supported on an inside surface of the gown, the device is referred to as a “patient warming gown.”

In one aspect, a patient warming gown may be worn by a person where it receives a stream of warmed pressurized air, distributes the pressurized air within a convective apparatus, and emits the air through one or more permeable walls of the convective apparatus to warm the person's body.

In another aspect, the patient warming gown may be employed to provide therapeutic warming during surgery. In this regard, the patient warming gown may be adapted for therapeutic warming by operating the convective apparatus for use intraoperatively while the clinical garment is adjusted so as to provide access to the surgical site.

In some aspects of the patient warming gown illustrated and discussed below, convective apparatuses are inflatable. That is, their structures, flaccid when not in use, tauten when receiving a stream of pressurized air.

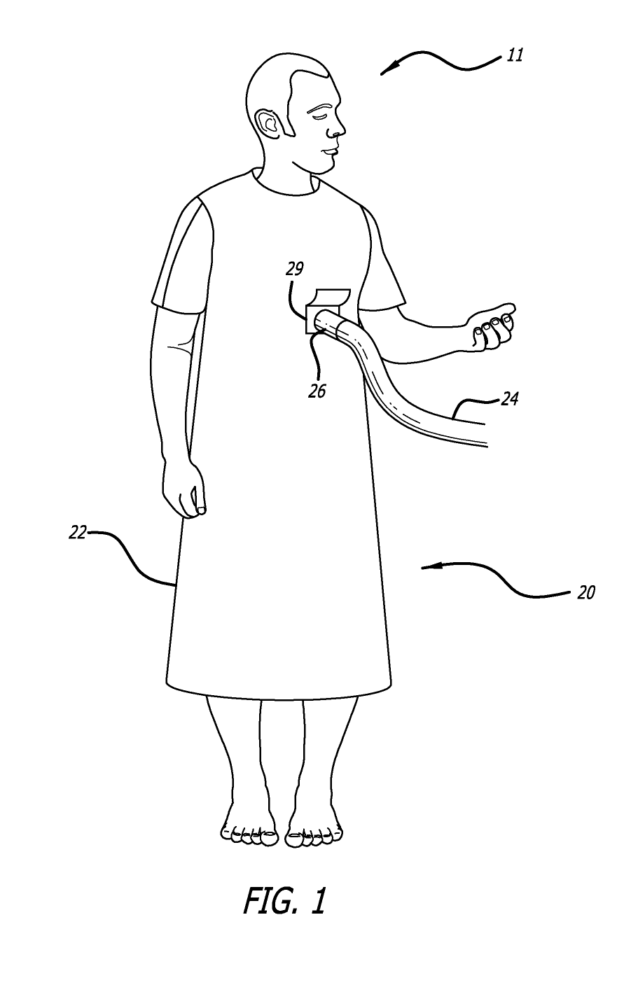

Refer now to the figures, in which a person 11 wearing a patient warming gown 20 with provision for peripheral warming is illustrated in FIG. 1. The warming device 20 is constituted of a clinical garment 22 and an inflatable convective apparatus (not seen in this view) integrated with or supported on a surface of the clinical garment 22. The convective apparatus may be operated by receiving warmed, pressurized air from a heater/blower unit (not seen in this view) through an air hose 24 with a nozzle 26 that is received in an inlet port of the convective apparatus. The inlet port is accessed through an opening 29 in the clinical garment 22. Other inlet ports (not shown) may be accessed through other openings in the clinical garment (not shown).

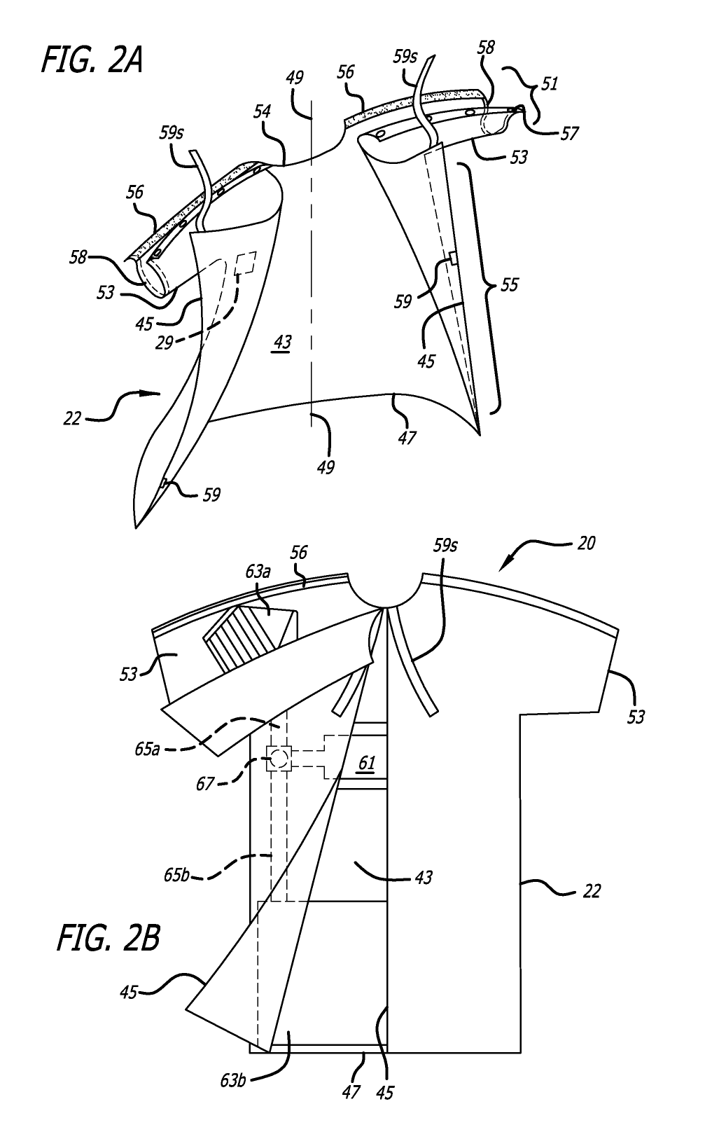

FIG. 2A illustrates the clinical garment element of a patient warming gown; an assembled patient warming gown 20 is itself illustrated in FIG. 2B. The view in FIGS. 2A and 2B is toward the inside surface of a clinical garment 22, which faces the chest, or thorax of a patient and on which an inflatable convective apparatus is mounted. With reference to FIGS. 2A and 2B, the patient warming gown 20 includes the clinical garment 22. The clinical garment 22 includes an inside surface 43, two opposing lateral hems 45 and a lower hem 47. A longitudinal axis 49 of the clinical garment is shown for reference. The clinical garment 22 has an upper portion 51 with two opposing sleeves 53 and an upper edge 54, and a lower portion 55. The opening 29 through the front of the garment 22 is also visible in FIG. 2A. The sleeves 53 may be long or short. Short sleeves are preferred if access must be had to a person's arms for instrumentation and/or IV delivery. Each sleeve 53 may include an elongate seam 56. Each seam 56 may be held closed by a sleeve closure element 57 including, for example, buttons, snaps, hook and loop material, tape, and/or straps, or any equivalent thereof. Such a sleeve closure element can be operated to let a seam 56 be opened and to again close a seam, once opened. Cuffs 58 may be formed in the clinical garment 22 inside the sleeves 53, near the ends of the sleeves 53. The clinical garment 22 may open on a side. Preferably, the clinical garment 22 opens in the rear. The opening may be full, as illustrated in FIG. 2A, or it may be a slit rising from the lower hem 47. As per the example shown in FIG. 2A, the opening may be closed by a rear closure element 59 along the lateral hems 45 which releasably connect to keep the hems together. Such a rear closure element may include, for example, buttons, snaps, hook and loop material, tape, and/or straps, or any equivalent thereof. In keeping with the example of FIG. 2A, if straps are used to close the opening, the straps may be attached to the clinical garment 22, or formed integrally therewith as a step in manufacturing the clinical garment 22. For example only, two integrally-formed straps 59s for tying the opposing lateral hems 45 together in the upper portion 51 are seen in FIG. 2A. Additional straps may be attached to the outside surface of the clinical garment 22 with enough reach to be tied together around the outside of the clinical garment, near its middle. With enough overlap of the lateral hems 45, the opening in the clinical garment 22 can be completely closed and secured, with the lateral hems overlapping to afford concealment of a patient's private parts. The clinical garment 22 may be constructed from non-woven or woven materials. Preferably, the clinical garment 22 is made from a non-woven blend of spunlace polyester and wood pulp

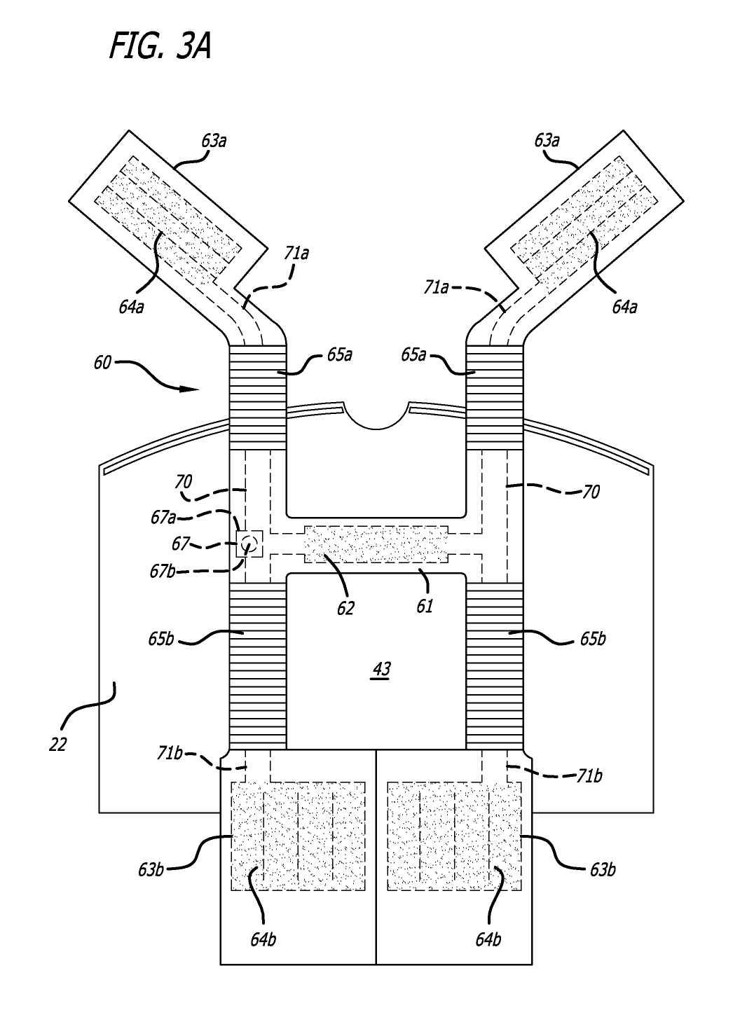

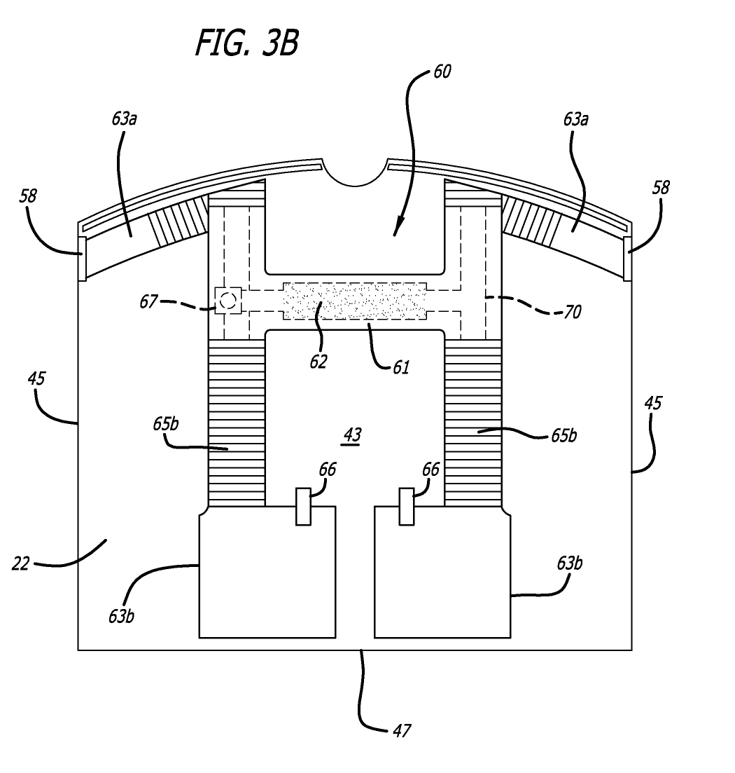

FIGS. 3A and 3B are plan views of an uninflated convective apparatus 60. In FIG. 3A, a convective apparatus 60 is shown fully opened for deployment to provide therapeutic warming. In FIG. 3B, elements of the convective apparatus 60 are shown folded. The view in both figures is toward a permeable structure that faces a person clothed in the patient warming gown 20. As seen in these figures, the convective apparatus 60 includes a central member 61, one or more peripheral diffusers 63, one or more flexible ducts 65 and one or more inlet ports 67. Elements of the convective apparatus 60 may be described as “folded” or in a “folded configuration;” which is also to include “rolled” or in a “rolled up configuration” or otherwise contracted configurations commonly known.

With reference to FIGS. 3A, and 3B, the central member 61 has a generally quadrilateral shape and an inflatable structure. In some aspects, but not necessarily, the inflatable structure may include an air-permeable wall 62 through which air is expelled when the member 61 is inflated. In some aspects related to FIGS. 3A and 3B, peripheral diffusers 63 include peripheral diffusers 63a for warming upper limbs (such as arms and/or hands) and peripheral diffusers 63b for warming lower limbs (such as legs and/or feet). In these aspects, each peripheral diffuser 63 has a generally quadrilateral shape and an inflatable structure that includes an air-permeable wall 64 through which air is expelled when the diffuser is inflated. Each duct 65 connects a respective peripheral diffuser 63 with the central member 61. When pressurized air flows into the central member 61, and the peripheral diffusers are deployed for use, the central member 61 inflates, pressurized air flows from the central member 61 through airflow channels 70 to the ducts which inflate and conduct the pressurized air to the peripheral diffusers 63. The pressurized air flows to and within airflow channels 71 to the peripheral diffusers 63, which inflate in response to the pressurized air flowing through the ducts 65.

In response to inflation, pressurized air is emitted through the air-permeable walls 62 and 64.

In some aspects, the convective apparatus 60 is configured prior to use as shown in FIGS. 2B and 3B. In this regard, opposing sides of the peripheral diffusers 63 may be folded toward each other, and then folded again in half. The folds reduce each diffuser 63 to a configuration that fits into the clinical garment 22. In some aspects each folded diffuser of one pair of peripheral diffusers 63a fits into a respective sleeve 53 of the clinical garment 22. The folds are preferably made so as to be easily tucked between the clinical garment 22 and the pair of ducts 65a that are connected to the diffusers 63a. The ends of the folded configurations may be retained in the inside cuffs 58 in the sleeves of the clinical garment 22 (best seen in FIG. 2B). This allows the patient to insert an arm through the sleeve of the clinical garment 22 without catching the corresponding hand on the fold and inadvertently deploying the extended side. Generally, the diffusers of the one pair of diffusers 63a may be folded, rolled or gathered in any way that achieves the desired length reduction and neat compaction useful for stowing and retention of the peripheral diffusers 63a, and unfolding them for deployment to the arms of a person. In this manner, the peripheral diffusers 63a are releasably retained on the clinical garment, preferably in a folded configuration, inside the sleeves 53.

In some aspects each folded diffuser of a second pair of peripheral diffusers 63b fits into an area of the inside surface 43, above the lower hem 47 of the clinical garment 22. The folded peripheral diffusers 63b may be retained by, for example, tape 66 acting between inside surface 43 and the folded diffusers. The folds are preferably made so as to position the diffusers 63b between the lower hem 47 and the pair of ducts 65b that are connected to the diffusers. In this manner, the peripheral diffusers 63b are releasably retained on the clinical garment, preferably inside a lower portion of the garment 22, on the lower surface. Generally, the diffusers of the pair of diffusers 63b may be folded, rolled or gathered in any way that achieves the desired length reduction and neat compaction useful for stowing and retention of the diffusers 63b, and unfolding them for deployment to the legs of a person. In this manner, the peripheral diffusers 63b are releasably retained on the clinical garment, preferably in a folded configuration, on a lower portion of the inside surface 43.

With reference to FIGS. 3A and 3B, although one inlet port 67 is illustrated in the convective apparatus 60, one or more additional inlet ports 67 may be provided for convenience. Unused inlet ports are sealed or closed by known means to prevent air escaping therethrough. Preferably the inlet port 67 is provided through a surface of the central member 61 which is not visible in these figures; it may also be provided through an edge of the central member 61. Per FIG. 3A, the inlet port 67 may comprise a collar 67a of stiff material with an opening 67b to receive the nozzle of an air hose, as taught in U.S. Pat. No. 7,244,268, or it may comprise a sleeve of material, or any other equivalent structure.

With reference to FIGS. 3A and 3B, the convective apparatus 60 is preferably constituted of flexible parts which are assembled to form inflatable structures in the central member 61, the diffusers 63, and the ducts 65, with air-permeable walls in the central member 61 and the diffusers 63. In response to a stream of pressurized air flowing into the convective apparatus 60 through an inlet port 67, the central member 61 inflates and emits air through the air-permeable wall 62 into the interior of the clinical garment 22. If the diffusers 63 are retained in folded configurations as shown in FIG. 3B, most, if not all of the pressurized air flowing into the convective apparatus 60 remains in and is emitted from the inflated central member 61. When a diffuser 63 is unfolded as shown in FIG. 3A, air flows from the central member 61, through a duct 65 to the diffuser 63, inflating both of those parts and being emitted through an air-permeable wall 71 in the diffuser 63.

An inflatable convective device 60 shown in the figures and described herein may be constructed using techniques and materials which are known in the art, or which are equivalent thereto. The specific details of construction and materials that are described are meant for illustration only. The convective apparatus 60 is formed or assembled separately from the clinical garment and then attached to its inside surface by tape, sewing, gluing, heat sealing, hook-and-loop, adhesion, stiction, or welding, or any combination of these. Generally, the construction and materials with which the central member 61 and peripheral diffusers 63 of the inflatable convective device 60 may be made include two or more sheets of flexible material that are brought together and bonded, joined, or sealed at a periphery to form inflatable space. Within the peripheral seals, the shapes and airflow characteristics of the inflatable space are established by stake points, and/or elongate seals. The sheets of material of which these elements are formed may be made of synthetic or natural materials, or a natural/synthetic blend. The sheet or sheets forming one side of an inflatable structure are made, or processed, to form an inflatable structure having an air-permeable wall with a surface through which pressurized air that inflates the structure, circulates, passes or exits toward the body of a person clothed in the clinical garment. Inlet ports, with provision for retention of an air hose nozzle, are provided for admitting a stream of warmed, pressurized air into the pneumatic structure, from an air hose connected to a heater/blower unit. The warmed air circulating through the surface causes the person to be warmed. Although convection is the principal mode of warming, the structure itself, when inflated with warmed air, radiates heat as well. Further, where the structure contacts the person's body, heat may also be transferred by conduction. Illustrative examples of inflatable thermal device construction and operation are described in U.S. Pat. Nos. 7,520,889 and 7,871,428. Illustrative examples of heater/blower construction and operation are described in U.S. Pat. Nos. 6,876,884; 7,819,911; and 7,976,572. While a convective device can be supported on an inner surface of the garment, as illustrated in FIGS. 2A-2B, the convective device can be integrated with the garment. For example, the garment can be air permeable and the convective device can be a structure attached at the outer surface of the garment, such that the convective device and the garment form one or more inflatable sections together.

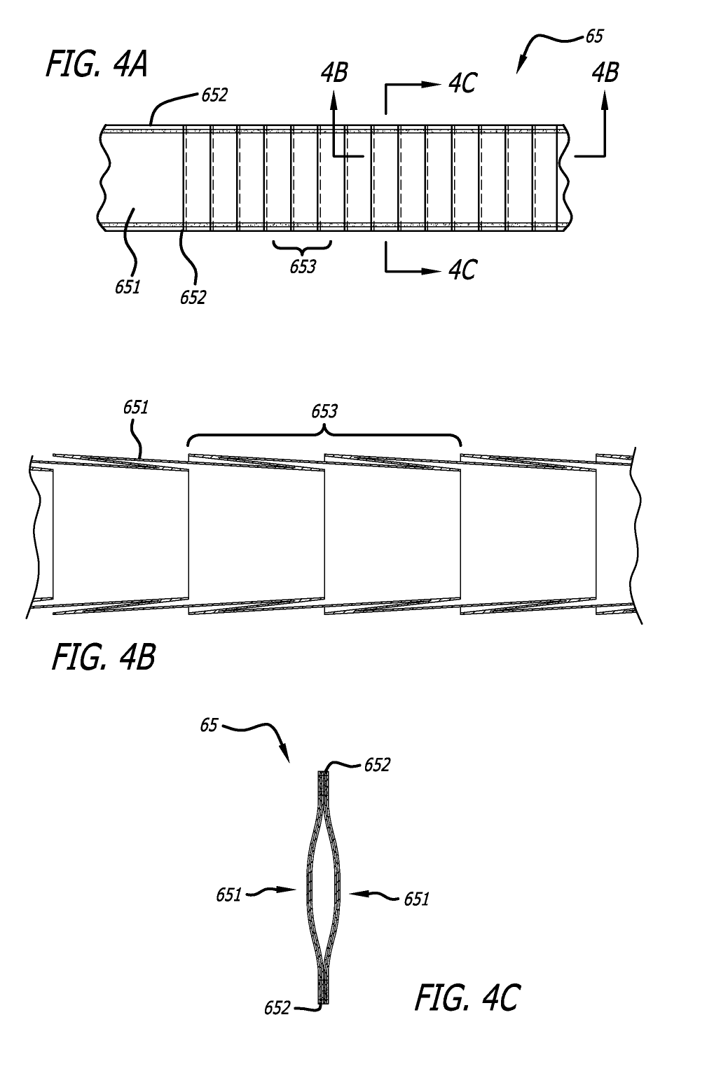

With reference to FIGS. 4A, 4B, and 4C, each duct 65 is constructed with two elongated sheets 651 of flexible material, in each of which a longitudinal sequence of Z-folds 653 is formed. The Z-fold sequences are aligned and the sheets are brought together and bonded, joined, or sealed along their long sides 652 to form inflatable space. Without inflation, the duct has a generally flat or planar configuration. When pressurized air flows into and through the space defined between the joined sheets, the duct inflates and the aligned Z-folds operate to form a longitudinal sequence of circumferential pleats along the duct. In this regard, the duct is circumferentially pleated, that is to say, each of the pleats runs in a generally circumferential direction of the duct. Such pleating imparts flexibility to the duct that aids in maintaining kink-free patency while it bends in simple or complex orientations. The sheets of material of which these ducts are formed may be made of synthetic or natural materials, or a natural/synthetic blend, as with the central member 61 and the diffusers 63. It is preferred that the ducts 65 be non-permeable, although in some instances it may be useful to provide the ducts with some degree of air permeability.

In some aspects, the elements 61, 63, and 65 of the convective device 60 are constructed as separate pieces and then joined together by bonding, joining, or sealing. In other aspects, automated manufacturing techniques and machines may be adapted, built, developed, and/or assembled to continuously form convective devices, and to join them with clinical garments. See U.S. Pat. No. 8,192,475 in this regard.

FIGS. 1 and 5-7 illustrate methods with which the patient warming gown 20 is used for warming the periphery of a person during a perioperative sequence. In FIG. 1, the patent is clothed in the patient warming gown 20 as would occur in a pre-induction facility where the patient is prepared for a surgical procedure. In this phase of surgical treatment, the patient can be warmed by inflation of the central member 61 with warmed pressurized air, without deployment of the peripheral diffusers 63. With the diffusers 63 retained in their folded configurations they provide little, if any warming effect. In some other aspects of pre-induction, since the peripheral parts of the body (arms and legs) represent the sites of greatest energy storage in patients prior to induction of anesthesia, the peripheral diffusers may be deployed during the preinduction phase to accelerate the prewarming process.

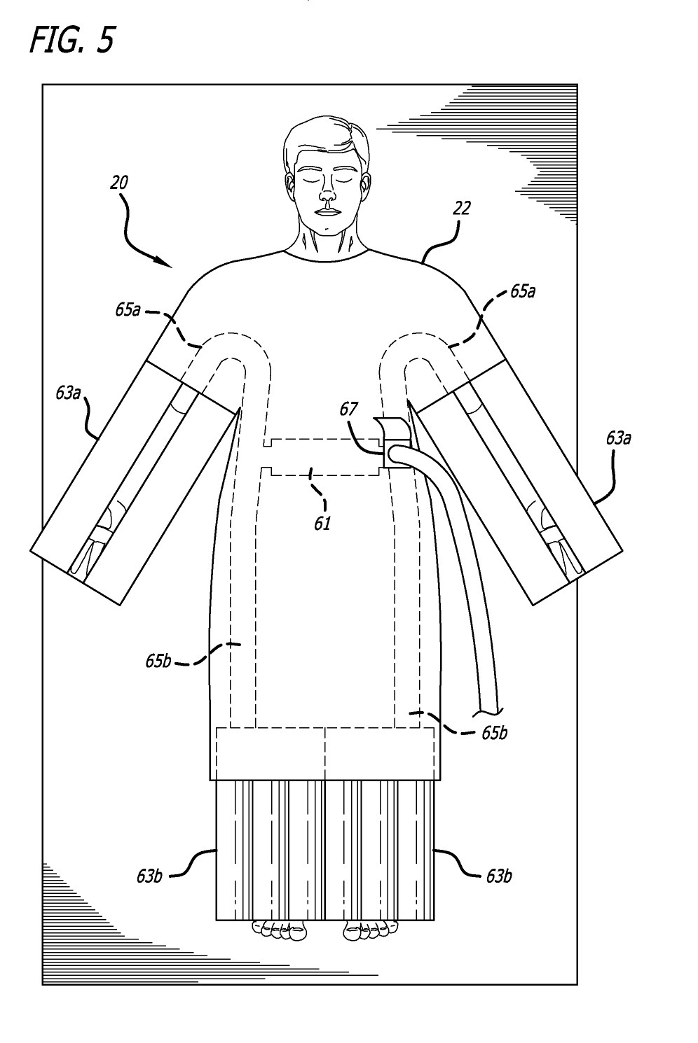

Various phases of the perioperative cycle are shown in FIGS. 5-7, where the view is toward the front of the clinical garment 22, from a position above the patient, who is lying prone on a surface such as an operating table or a gurney (neither shown). As seen in FIGS. 5-7 all four diffusers are deployed for respective examples to be described. However, this is not meant to limit the methods of use, some of which might need only one, two, or three diffusers to be deployed for use.

In FIG. 5, a patient is shown wearing a patient warming gown 20 as preparation for surgery commences. The clinical garment 22 is loosened and one or more of the diffusers 63a and 63b is unfurled. When unfurled, the diffusers 63a are aligned with and engage the patient's arms, with their air-permeable walls facing the arms. The diffusers 63b are aligned with and engage the patient's legs, with their air-permeable walls facing the legs. The inlet port 67 is accessed and a heater/blower unit (not shown) is connected to the inlet port 67 via an air hose and nozzle. Warmed pressurized air flows into and inflates the central member 61. From the central member 61, the pressurized air flows through and inflates the ducts 65 and then the peripheral diffusers 63. While the central member 61 continues to warm the patient's upper torso, the unfurled diffusers 63 are now operative to provide peripheral warming to the patient by diffusing and circulating warmed air to the patient's extremities.

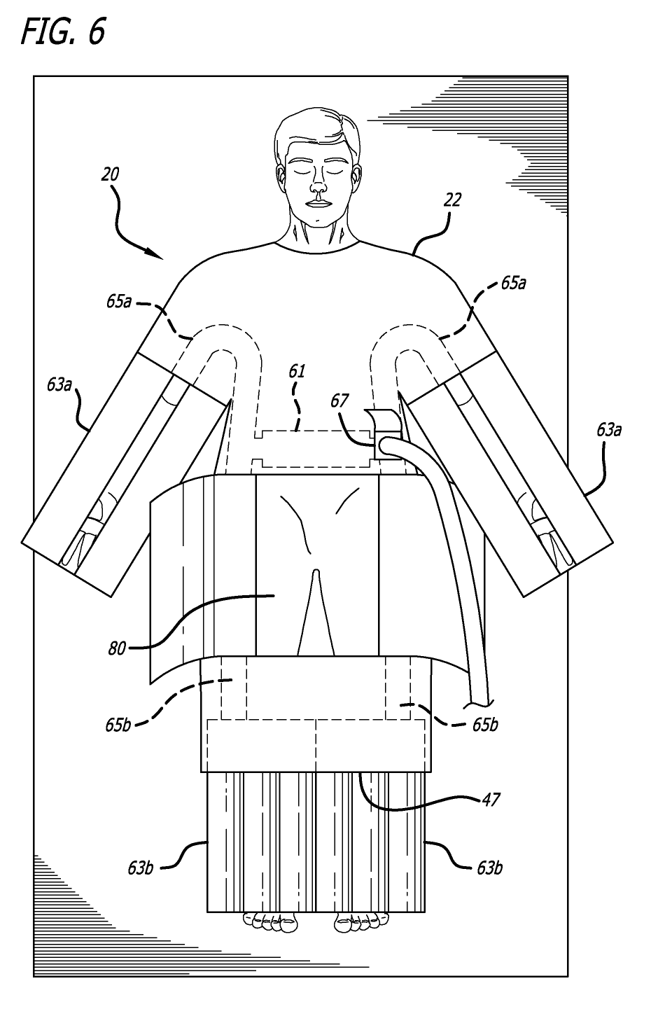

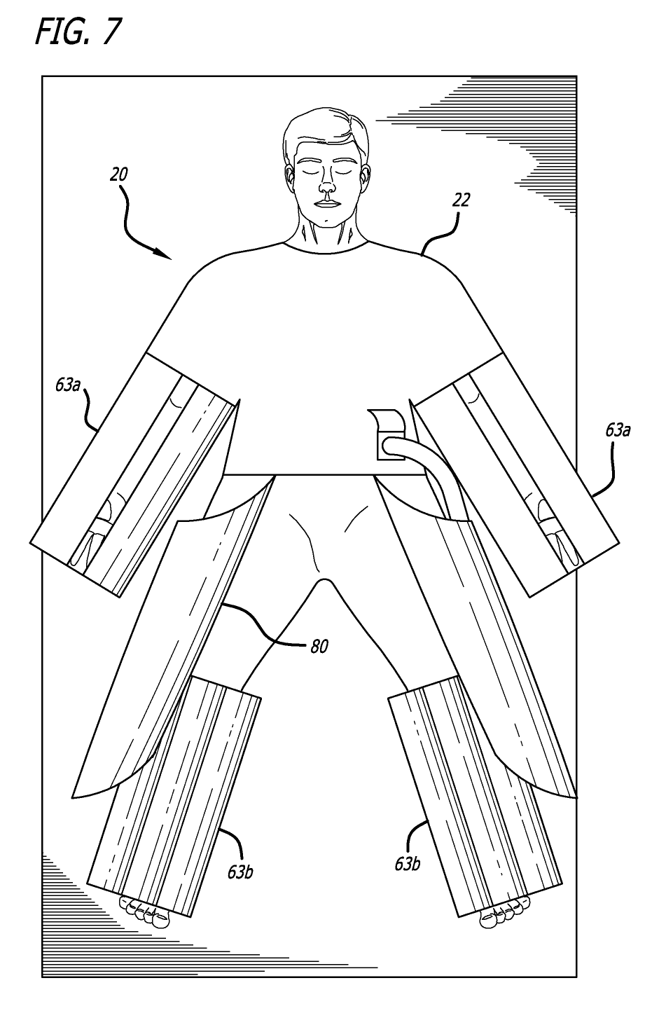

In FIG. 6, an opening 80 is formed in the lower portion of the clinical garment beneath the central member 61 in a generally axial direction toward the lower hem 47 of the clinical garment. As seen in FIG. 6, both pairs of diffusers are fully unfurled for peripheral warming of the patient while the opening 80 in the patient warming gown provides unobstructed access to a surgical site in the abdominal and/or pelvic areas of the patient. FIG. 7 shows the opening 80 after having been enlarged by splitting the lower section 81 to the lower hem 47. This permits utilization of known patient configurations such as Fowler positions (shown in FIG. 7), lithotomy positions, and other positions needed for pelvic and abdominal procedures.

The openings 80 in the clinical garment 22 that are seen in FIGS. 6 and 7 can be made by cutting the garment during the initial stages of surgical preparation. Preferably, but not necessarily, these and other openings can be predefined by patterns of perforation made in the clinical garment 22 during the manufacturing process.

During the post-operative phase, the openings 80 can be closed by tape so as to retain warmed air around the patient, and the patient can be warmed by operation of the central member 61 and any one or more of the peripheral diffusers 63.

Manifestly, the ducted construction of the convective device 60 provides significant convenience during the intra-operative phase. The lateral positioning of the ducts 63b with respect to the axis 49 of the clinical garment affords unimpeded access through the clinical garment to the patient's abdominal, pelvic, and upper thigh areas. Separation of the constructions of the diffusers 63 and ducts 65 from the clinical garment provides great flexibility in positioning those elements with respect to the clinical garment and the locations of the patient's limbs during and after surgery. The pleated ducts permit the peripheral diffusers to be moved in several planes of rotation so that the diffusers remain inflated.

### EXEMPLARY EMBODIMENTS

Embodiment 1. A warming device, comprising: a clinical garment having a surface; and, an inflatable convective warming apparatus on the surface; wherein, the convective warming apparatus includes an inflatable central member attached to the surface, at least one inlet port, and at least one permeable peripheral diffuser that is separate from the clinical garment and coupled to the central member by a pleated, non-kinking duct.

Embodiment 2. The warming device of Embodiment 1, wherein the at least one permeable peripheral diffuser is releasably retained in a folded, rolled, or contracted configuration on the clinical garment.

Embodiment 3. The warming device of Embodiment 1 or 2, wherein the at least one permeable peripheral diffuser and the duct are inflatable.

Embodiment 4. The warming device of Embodiment 3, wherein the central member and the at least one peripheral diffuser have respective air-permeable walls.

Embodiment 5. The warming device of Embodiment 4, wherein the duct is non-permeable.

Embodiment 6. The warming device of any one of Embodiment 1 to Embodiment 5, wherein the at least one permeable diffuser includes at least two permeable diffusers, each coupled to the central member by a respective non-permeable duct, and each positioned on the clinical garment to align with a respective arm of a patient.

Embodiment 7. The warming device of any one of Embodiment 1 to Embodiment 6, wherein the at least one permeable diffuser includes at least two permeable diffusers, each coupled to the central member by a respective non-permeable duct, and each positioned on the clinical garment to align with a respective leg of a patient.

Embodiment 8. A warming device, comprising: a clinical garment having a surface; and, an inflatable convective warming apparatus on the surface; wherein, the convective warming apparatus includes an inflatable central member attached to the surface, at least one inlet port, at least one pair of air-permeable peripheral diffusers that are detachable from the clinical garment, and at least one pair of circumferentially pleated ducts, each duct connecting a respective air-permeable diffuser to the central member.

Embodiment 9. The warming device of Embodiment 8, wherein each air-permeable peripheral diffuser is releasably retained in a folded configuration on the clinical garment.

Embodiment 10. The warming device of Embodiment 8 or Embodiment 9, wherein the air-permeable peripheral diffusers and the ducts are inflatable.

Embodiment 11. The warming device of Embodiment 10, wherein the central member and the air-permeable peripheral diffusers have respective air-permeable walls.

Embodiment 12. The warming device of Embodiment 11, wherein the ducts are non-permeable.

Embodiment 13. The warming device of any one of Embodiment 8 to Embodiment 12, wherein the air-permeable diffusers are positioned on sleeves of the clinical garment to align with the arms of a patient.

Embodiment 14. The warming device of any one of Embodiment 8 to Embodiment 13, wherein the air-permeable diffusers are positioned near a lower hem of the clinical garment to align with the legs of a patient.

Embodiment 15. The warming device of any one of Embodiment 8 to Embodiment 14, wherein the at least one pair of air-permeable peripheral diffusers includes first and second pairs of air-permeable peripheral diffusers that are separable from the clinical garment, and the at least one pair of ducts includes first and second pairs of ducts, in which each duct of the first pair of ducts connects a respective air-permeable diffuser of the first pair of air-permeable peripheral diffusers to the central member; and, each duct of the second pair of ducts connects a respective air-permeable diffuser of the second pair of air-permeable peripheral diffusers to the central member.

Embodiment 16. The warming device of Embodiment 15, wherein the first pair of air-permeable diffusers are positioned on sleeves of the clinical garment to align with the arms of a patient and the second pair of air-permeable diffusers are positioned near a lower hem of the clinical garment to align with the legs of a patient.

Embodiment 17. A method of operating a warming apparatus including a clinical garment with an inflatable convective warming apparatus on a surface of the clinical garment in which the convective warming apparatus includes an inflatable central member attached to the surface, at least one inlet port, and at least one permeable peripheral diffuser coupled to the central member by a duct, comprising: disposing the clinical garment on a person; deploying the permeable peripheral diffuser on a leg of the person; and providing a stream of warmed, pressurized air into the central member.

Embodiment 18. The method of Embodiment 17, wherein the permeable peripheral diffuser is releasably retained on the clinical garment and deploying the permeable peripheral diffuser includes releasing the permeable peripheral diffuser from the clinical garment, and placing the permeable peripheral diffuser on the leg.

Embodiment 19. A method of operating a convective warming apparatus including a clinical garment with an inflatable convective warming apparatus on a surface of the clinical garment, in which the convective warming apparatus includes a permeable central member attached to the surface, at least one inlet port in the central member, and at least one permeable peripheral diffuser coupled to the central member by a duct, comprising: retaining the permeable peripheral diffuser in a folded configuration in the clinical garment; disposing the clinical garment on a person; unfolding the permeable peripheral diffuser; and, placing the permeable peripheral diffuser over a leg of the person.

Embodiment 20. The method of Embodiment 19, further including connecting an air hose to the inlet port, and providing a flow of heated air through the air hose.

Various modifications and alterations to this invention which do not depart from its scope and spirit will become apparent to those skilled in the art. Consequently, it should be understood that this invention is not intended to be unduly limited by the illustrative embodiments and examples set forth herein and that such examples and embodiments are presented by way of example and that the scope of the invention is intended to be limited only by the claims set forth herein as follows.

1. A warming device, comprising:
a clinical garment having a surface; and,
an inflatable convective warming apparatus on the surface; wherein,
the convective warming apparatus includes an inflatable central member attached to the surface, at least one inlet port, and at least one permeable peripheral diffuser that is separate from the clinical garment and coupled to the central member by a pleated, non-kinking duct.

2. The warming device of claim 1, wherein the at least one permeable peripheral diffuser is releasably retained in a folded, rolled, or contracted configuration on the clinical garment.

3. The warming device of claim 1, wherein the at least one permeable peripheral diffuser and the duct are inflatable.

4. The warming device of claim 3, wherein the central member and the at least one peripheral diffuser have respective air-permeable walls.

5. The warming device of claim 4, wherein the duct is non-permeable.

6. The warming device of claim 1, wherein the at least one permeable diffuser includes at least two permeable diffusers, each coupled to the central member by a respective non-permeable duct, and each positioned on the clinical garment to align with a respective arm of a patient.

7. The warming device of claim 1, wherein the at least one permeable diffuser includes at least two permeable diffusers, each coupled to the central member by a respective non-permeable duct, and each positioned on the clinical garment to align with a respective leg of a patient.

8. A warming device, comprising:
a clinical garment having a surface; and,
an inflatable convective warming apparatus on the surface; wherein,
the convective warming apparatus includes an inflatable central member attached to the=surface, at least one inlet port, at least one pair of air-permeable peripheral diffusers that are detachable from the clinical garment, and at least one pair of circumferentially pleated ducts, each duct connecting a respective air-permeable diffuser to the central member.

9. The warming device of claim 8, wherein each air-permeable peripheral diffuser is releasably retained in a folded configuration on the clinical garment.

10. The warming device of claim 8, wherein the air-permeable peripheral diffusers and the ducts are inflatable.

11. The warming device of claim 10, wherein the central member and the air-permeable peripheral diffusers have respective air-permeable walls.

12. The warming device of claim 11, wherein the ducts are non-permeable.

13. The warming device of claim 8, wherein the air-permeable diffusers are positioned on sleeves of the clinical garment to align with the arms of a patient.

14. The warming device of claim 8, wherein the air-permeable diffusers are positioned near a lower hem of the clinical garment to align with the legs of a patient.

15. The warming device of claim 8, wherein the at least one pair of air-permeable peripheral diffusers includes first and second pairs of air-permeable peripheral diffusers that are separable from the clinical garment, and the at least one pair of ducts includes first and second pairs of ducts, in which
each duct of the first pair of ducts connects a respective air-permeable diffuser of the first pair of air-permeable peripheral diffusers to the central member; and,
each duct of the second pair of ducts connects a respective air-permeable diffuser of the second pair of air-permeable peripheral diffusers to the central member.

16. The warming device of claim 15, wherein the first pair of air-permeable diffusers are positioned on sleeves of the clinical garment to align with the arms of a patient and the second pair of air-permeable diffusers are positioned near a lower hem of the clinical garment to align with the legs of a patient.

17. A method of operating a warming apparatus including a clinical garment with an inflatable convective warming apparatus on a surface of the clinical garment in which the convective warming apparatus includes an inflatable central member attached to the surface, at least one inlet port, and at least one permeable peripheral diffuser coupled to the central member by a duct, comprising:
disposing the clinical garment on a person;
deploying the permeable peripheral diffuser on a leg of the person; and
providing a stream of warmed, pressurized air into the central member.

18. The method of claim 17, wherein the permeable peripheral diffuser is releasably retained on the clinical garment and deploying the permeable peripheral diffuser includes releasing the permeable peripheral diffuser from the clinical garment, and placing the permeable peripheral diffuser on the leg.

19. A method of operating a convective warming apparatus including a clinical garment with an inflatable convective warming apparatus on a surface of the clinical garment in which the convective warming apparatus includes a permeable central member attached to the inside surface, at least one inlet port in the central member, and at least one permeable peripheral diffuser coupled to the central member by a duct, comprising:
retaining the permeable peripheral diffuser in a folded configuration in the clinical garment;
disposing the clinical garment on a person;
unfolding the permeable peripheral diffuser; and,
placing the permeable peripheral diffuser over a leg of the person.

20. The method of claim 19, further including connecting an air hose to the inlet port, and providing a flow of heated air through the air hose.
